(19) United States
(12) Patent Application Publication
(10) Pub. No.: US 2025/0302802 A1
(43) Pub. Date: Oct. 2, 2025

(54) METHODS AND COMPOSITIONS

(71) Applicant: VRG 145 ZRT., Budapest (HU)

(72) Inventors: Sándor FARKAS, Budapest (HU); Csilla HORVÁTH, Budapest (HU); Csongor Béla CSEKO, Budapest (HU); Éva MAKÓ, Budapest (HU); Istvánné KIS-VARGA, Kakucs (HU); Tamás KITKA, Kerepes (HU)

(21) Appl. No.: 19/092,880

(22) Filed: Mar. 27, 2025

Related U.S. Application Data
(60) Provisional application No. 63/570,515, filed on Mar. 27, 2024.

(51) Int. Cl. A61K 31/40; A61K 31/13; A61P 25/06

(52) U.S. Cl. CPC A61K 31/40; A61K 31/13; A61P 25/06

(57) ABSTRACT

The disclosure provides new and improved combination therapies using lisinopril and memantine.

### METHODS AND COMPOSITIONS

Migraine is a complex and relatively common neurological condition that is characterized by severe, episodic attacks of headache in concert with associated symptoms, which may include nausea, vomiting, and sensitivity to light, sound, and/or movement. The headache pain may be severe, and migraines have been found to be associated with psychiatric and medical comorbidities such as depression and vascular disorders. Migraine attacks are highly disruptive and cost billions of dollars each year in missed workdays and impaired work performance.

Current approved migraine pharmacotherapies for acute treatment include lasmiditan, the triptan class of drugs, and calcitonin gene-related peptide (CGRP) receptor antagonists, also known as “gepants”, which include ubrogepant and rimegepant. Therapies for preventive treatment include beta blockers (such as propranolol or timolol), anticonvulsants (such as topiramate or sodium valproate), and a large variety of off-label drugs. Recently, anti-CGRP antibodies and CGRP antagonists (atogepant, rimegepant) have seen interest as potential prophylactic treatments for migraines.

However, each of these therapeutics pose challenges. Triptans are contraindicated in a portion of patients, particularly those with ischemic cardiovascular diseases. Additionally, 30% of migraine attacks are triptan resistant, and newer agents such as lasmiditan, rimegepant, and ubrogepant showed lower odds of achieving pain freedom or pain relief compared with triptans. On the prophylactic side, current therapies are only partially effective and are associated with numerous side effects. The wide variety of therapies and the trial-and-error manner by which a patient's protocol is determined suggests a lack of effective solutions for the treatment of migraine. Treatment resistance may also be significantly higher in both conventional treatments (e.g. topiramate and propranolol) and newer CGRP-targeting agents.

Therefore, there exists a need for novel and improved therapies for the treatment of migraine. This disclosure provides combination therapies using lisinopril and memantine.

Citation of any reference throughout this application is not to be construed as an admission that such reference is prior art to the present application.

### SUMMARY

Provided herein is a pharmaceutical composition comprising lisinopril, or a pharmaceutically acceptable salt thereof, and memantine, or a pharmaceutically acceptable salt thereof, and optionally a pharmaceutically acceptable excipient.

Also provided is a kit comprising a first container and a second container, the first container comprising lisinopril, or a pharmaceutically acceptable salt thereof, and the second container comprising memantine, or a pharmaceutically acceptable salt thereof.

Also provided is a method of alleviating or preventing pain, wherein the pain is selected from migraine, tension-type headache, medication overuse headache, trigeminal autonomic cephalalgia (TAC), and trigeminal neuralgia, comprising administering to a subject in need thereof a composition or kit as described herein.

Also provided is a compound or kit as described herein for use in the treatment of pain, wherein the pain is selected from migraine, tension-type headache, medication overuse headache, trigeminal autonomic cephalalgia (TAC), and trigeminal neuralgia.

These and other aspects of the disclosure will be apparent upon reference to the following detailed description. To this end, various references are set forth herein which describe in more detail certain background information, procedures, compounds, and/or compositions, and are each hereby incorporated by reference in their entirety.

### BRIEF DESCRIPTION OF THE DRAWINGS

FIG. 1 depicts a semilog plot showing dose-response relationships for lisinopril, memantine, and their 1:1 ratio combination where the effects are expressed in percent reversal of allodynia in rats. The doses of the combination were plotted at doses of its individual components. Data are presented as mean±S.E.M. of N=6-11 per group. Calculated ED₅₀ values are also indicated.

FIG. 2 depicts an isobologram with points in a coordinate system representing ED₅₀ values expressed as doses of two individual components of a combination in rats. The isobole (dashed line) marks the points of the coordinate system where there are combinations of additive interaction. The dotted line indicates 1:1 combinations. The half-filled circle indicates the theoretical point of an additive combination. Points representing synergistic combinations fall below the isobole (closer to origo); subadditive combinations fall above the isobole (more distant from origo).

FIG. 3 depicts the average curve of drug effects in rats over time at low (top graph) mid (middle graph) and high (bottom graph) doses of vehicle, lisinopril, memantine, and the lisinopril:memantine 1:1 ratio combination. Data are presented as mean±S.E.M.

FIG. 4 depicts a dose-response graph for lisinopril, memantine, and the lisinopril:memantine 1:1 ratio combination in rats. Data of 90 minutes post-dose were converted to percent reversals and plotted as mean±S.E.M.

FIG. 5 depicts graphs showing the effects over time on mean arterial pressure and heart rate before and after doses of vehicle, lisinopril, memantine, and the lisinopril:memantine 1:1 ratio combination. Data are presented as mean±S.E.M.

FIG. 6 depicts a bar graph showing the effects of doses of vehicle, lisinopril, memantine, and the lisinopril:memantine 1:1 ratio combination on horizontal locomotor activity of rats. Data are presented as mean±S.E.M.

FIG. 7 depicts a bar graph showing the effects of doses of vehicle, lisinopril, memantine, and the lisinopril:memantine 1:1 ratio combination on rotarod drop-off time of rats.

FIG. 8 depicts a dose-response graph for candesartan, memantine, and the candesartan:memantine 1:1 ratio combination in rats. Data of 90 minutes post-dose were converted to percent reversals and plotted as mean±S.E.M.

### DETAILED DESCRIPTION OF THE DISCLOSURE

The definitions and methods provided define the present disclosure and guide those of ordinary skill in the art in the practice of the present disclosure. Unless otherwise noted, terms are to be understood according to conventional usage by those of ordinary skill in the relevant art.

As used herein, the terms “subject” or “patient” refer to a human. Unless otherwise noted, the terms “patient” or “subject” are used herein interchangeably.

As used herein, “treating” and the like mean the administration of therapy to a subject who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder. As related to the present disclosure, the term may also mean the administration of pharmacological substances or formulations, or the performance of non-pharmacological methods including, but not limited to, radiation therapy and surgery.

As used herein, “treatment” is an approach for obtaining beneficial or desired clinical results. For the purposes of this invention, beneficial or desired clinical results include, but are not limited to, one or more of the following: improvement in any aspect of headache, including lessening severity, alleviation of pain intensity, and other associated symptoms, reducing frequency of recurrence, reducing frequency of headache, increasing the quality of life of those suffering from the headache, and decreasing dose of other medications required to treat the headache. Using migraine as an example, other associated symptoms include, but are not limited to, nausea, vomiting, and sensitivity to light, sound, and/or movement.

As used herein, “acute treatment” is an approach for obtaining immediate beneficial or desired clinical results. For purposes of this invention, immediate beneficial or desired clinical results include, but are not limited to, one or more of the following: an increase in pain freedom and most bothersome symptom (MBS) freedom at two hours after dosing, wherein pain freedom can be defined as a reduction of moderate or severe headache pain to no headache pain and MBS freedom as the absence of the self-identified MBS, such as photophobia, phonophobia or nausea, an increase in pain relief at 2 hours, wherein pain relief can be defined as the reduction in migraine pain from moderate or severe severity to mild or none, an increase in sustained pain freedom at 2-48 hours, a reduction in the use of rescue medication within 24 hours, and an increase in the percentage of patients reporting normal function at two hours after dosing.

As used herein, “preventive treatment” is synonymous with the term “preventative treatment” and is an approach for obtaining beneficial or desired clinical results over time. For purposes of this invention, beneficial or desired clinical results over time include, but are not limited to, one or more of the following: an improvement in aspects of headache, including reducing frequency of recurrence, reducing frequency of headache, increasing the quality of life of those suffering from the headache, and decreasing dose of other medications required to treat the headache.

As used herein, “preventing” is an approach to stop headache from occurring or existing in a subject, who is susceptible to the development of headache. For example, the patient may have been previously diagnosed with chronic or episodic migraine.

“Reducing headache incidence” or “reducing headache frequency” means any of reducing severity (which can include reducing need for and/or amount of (e.g., exposure to) other drugs and/or therapies generally used for this headache condition), duration, and/or frequency (including, for example, delaying or increasing time to next headache attack in an individual). As is understood by those skilled in the art, individuals may vary in terms of their response to treatment, and, as such, for example, a “method of reducing frequency of headache in an individual” reflects administering an agent based on a reasonable expectation that such administration may likely cause such a reduction in headache incidence in that particular individual.

“Ameliorating” headache or one or more symptoms of headache means a lessening or improvement of one or more symptoms of headache as compared to not administering an agent. “Ameliorating” also includes shortening or reduction in duration of a symptom.

As used herein, “controlling headache” refers to maintaining or reducing severity or duration of one or more symptoms of headache or frequency of headache (e.g., migraine) attacks in an individual (as compared to the level before treatment). For example, the duration or severity of head pain, or frequency of attacks is reduced by at least about any of 10%, 20%, 30%, 40%, 50%, 60%, 70%, 80%, 90% or more, in the individual as compared to the duration or severity of head pain, or frequency of attacks before treatment.

As used herein, a “headache day” refers to a day during which a subject experiences headache. Headache days can be expressed in terms of whole days (e.g., one headache day, two headache days, three headache days, etc.) or in terms of whole and partial days (e.g., 0.5 headache days, 1.2 headache days, 2.67 headache days, etc.). One or more headache days may be described with respect to a particular time interval. For example, “weekly headache days” may refer to the number of headache days a subject experiences within a week interval (e.g., a 7-day period). As can be appreciated, a week interval may or may not correspond to a calendar week. In another example, “monthly headache days” may refer to the number of headache days a subject experiences within a month interval. As can be appreciated, a month interval (e.g., a period of 28, 29, 30, or 31 days) may vary in terms of number of days depending upon the particular month and may or may not correspond to a calendar month. In yet another example, “yearly headache days” may refer to the number of headache days a subject experiences within a year interval. As can be appreciated, a year interval (e.g., a period of 365 or 366 days) may vary in terms of number of days depending upon the particular year and may or may not correspond to a calendar year.

As used herein, “delaying” the development of headache means to defer, hinder, slow, retard, stabilize, and/or postpone progression of the disease. This delay can be of varying lengths of time, depending on the history of the disease and/or individuals being treated. As is evident to one skilled in the art, a sufficient or significant delay can, in effect, encompass prevention, in that the individual does not develop headache. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Development” or “progression” of headache means initial manifestations and/or ensuing progression of the disorder. Development of headache can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of headache includes initial onset and/or recurrence.

“Responder rate” means the proportion of patients reaching at least a 50% reduction in monthly average number of migraine days during a predetermined treatment period. In one embodiment of the invention, the predetermined treatment period is 3 months. In another embodiment of the invention, the predetermined treatment period is 6 months. In yet another embodiment of the invention, the predetermined treatment period is 12 months.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, reduction in responder rate or delaying the onset of the disease, including biochemical, histological and/or behavioral symptoms of the disease, its complications and intermediate pathological phenotypes presenting during development of the disease. For therapeutic use, beneficial or desired results include clinical results such as reducing pain intensity, duration, or frequency of headache attack, reduction in responder rate and decreasing one or more symptoms resulting from headache (biochemical, histological and/or behavioral), including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “subtherapeutic amount” refers to a dose of a therapeutic compound (e.g., an inhibitor) that is lower than the usual or typical dose of the therapeutic compound when administered alone for the treatment of a pain (e.g., migraine).

As used herein, the term “therapeutic amount” refers to the usual or typical dose of a therapeutic compound (e.g., an inhibitor) when administered alone for the treatment of a pain (e.g., migraine).

As used herein, the term “therapeutic index” refers to the ratio of the dose of a therapeutic compound that causes adverse effects at an incidence and/or severity incompatible with the targeted indication (e.g., a toxic dose in 50% of subjects, or TD₅₀) to the dose that leads to the desired pharmacological effect (e.g., an efficacious dose in 50% of subjects, or ED₅₀).

As used herein, the term “synergistic effect” refers to a combined therapeutic effect arising from an interaction between two or more drugs that is greater than the sum of the effects seen when each drug is given alone.

The compounds disclosed herein can exist as pharmaceutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “pharmaceutically acceptable salt,” as used herein, represents salts or zwitterionic forms of the compounds disclosed herein which are water or oil-soluble or dispersible and pharmaceutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

The compounds described herein according to the invention are also intended to include such compounds wherein the molecular structures include isotopes of carbon, hydrogen and nitrogen atoms occurring on those structures. Isotopes include those atoms having the same atomic number but different mass numbers. For example, isotopes of hydrogen include deuterium. Isotopes of carbon include ¹³C. Isotopes of nitrogen include ¹⁵N.

Accordingly, within the chemical structure of any chemical compound taught in this application as suitable for the formulations disclosed herein:
any hydrogen atom or group of hydrogen atoms, could suitably be replaced by an isotope of hydrogen, i.e., deuterium;
any carbon atom or group of carbon atoms, could suitably be replaced by an isotope of carbon, i.e., ¹³C; and
any nitrogen atom or group of nitrogen atoms, could suitably be replaced by an isotope of nitrogen, i.e., ¹⁵N.

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value, the range which would be included by rounding up or down to that figure as well taking into account significant figures, and the range which would encompass the recited value plus or minus 20%. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc., up to 5 hours.

The terms “a,” and “an,” and “the,” and similar references used in the context of describing a particular embodiment (especially in the context of certain of the following claims) can be construed to cover both the singular and the plural, unless specifically noted otherwise. The term “or” as used herein, including the claims, is used inclusively unless explicitly indicated to refer to alternatives only or the alternatives are mutually exclusive.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Provided herein is a pharmaceutical composition comprising lisinopril, or a pharmaceutically acceptable salt thereof, and memantine, or a pharmaceutically acceptable salt thereof, and optionally a pharmaceutically acceptable excipient.

In some embodiments, the pharmaceutical composition comprises a combination dosage form comprising lisinopril, or a pharmaceutically acceptable salt thereof, and memantine, or a pharmaceutically acceptable salt thereof, and optionally a pharmaceutically acceptable excipient. In some embodiments, the pharmaceutical composition comprises a kit comprising a first container and a second container, the first container comprising lisinopril, or a pharmaceutically acceptable salt thereof, and the second container comprising memantine, or a pharmaceutically acceptable salt thereof, each optionally also comprising a pharmaceutically acceptable excipient.

As used herein, “lisinopril” refers to an angiotensin-converting enzyme (ACE) inhibitor with a chemical formula of(S)-1-[N2-(1-carboxy-3-phenylpropyl)-L-lysyl]-L-proline and a structural formula of:

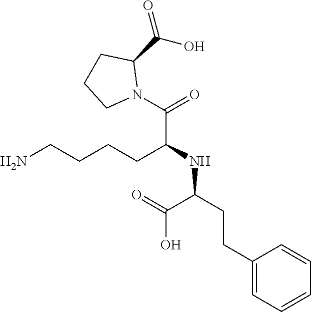 [SMILES: NCCCC[C@H](N[C@@H](CCc1ccccc1)C(=O)O)C(=O)N1CCC[C@H]1C(=O)O]

Lisinopril dihydrate, as contained in the U.S. FDA approved drug products such as ZESTRIL® or PRINIVIL® tablets which are supplied as 2.5 mg, 5 mg, 10 mg, 20 mg, 30 mg and 40 mg tablets and QBRELIS® solution which is supplied as a 1 mg/1 ml solution, or U.S. FDA approved AB rated generic equivalents thereof, for oral administration. In patients with uncomplicated essential hypertension not on diuretic therapy, the recommended initial dose is 10 mg once a day. Dosage should be adjusted according to blood pressure response. The usual dosage range is 20 to 40 mg per day administered in a single daily dose. The contents of the U.S. FDA approved package inserts for the forgoing lisinopril products are incorporated herein by reference.

In some embodiments, lisinopril, or a pharmaceutically acceptable salt thereof, is(S)-1-[N2-(1-carboxy-3-phenylpropyl)-L-lysyl]-L-proline dihydrate, (S)-1-[N2-(1-carboxy-3-phenylpropyl)-L-lysyl]-L-proline monohydrate, or (S)-1-[N2-(1-carboxy-3-phenylpropyl)-L-lysyl]-L-proline hydrochloride.

In some embodiments, pharmaceutical compositions of the disclosure include lisinopril, i.e., lisinopril as an unsolvated free base. In other aspects, pharmaceutical compositions of the disclosure include lisinopril as a solvated free base. In other aspects, pharmaceutical compositions of the disclosure include pharmaceutically acceptable salts of lisinopril. As used herein, amounts of lisinopril present in the pharmaceutical composition of the disclosure refer to amounts of lisinopril as an unsolvated free base. For example, in those aspects wherein the pharmaceutical composition comprises lisinopril free base, “2.5 mg of lisinopril” refers to 2.5 mg of the lisinopril unsolvated free base in the pharmaceutical composition. In aspects wherein the pharmaceutical composition comprises a solvate of lisinopril, such as lisinopril dihydrate, “2.5 mg of lisinopril” refers to 2.5 mg unsolvated lisinopril free base, based on 2.72 mg of lisinopril dihydrate in the pharmaceutical composition.

As used herein, “memantine” refers to 1-amino-3,5-dimethyladamantane and has a structural formula of:

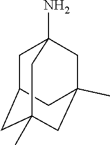

Memantine, as contained in the U.S. FDA approved drug products such as NAMENDA® which are supplied as 5 mg or 10 mg tablets and 2 mg/2 ml solution or as NAMENDA XR® which is supplied as 7 mg, 14 mg, 21 mg and 28 mg extended release capsules, or U.S. FDA approved AB rated generic equivalents thereof, for oral administration. The recommended starting dose of the tablets is 5 mg once daily. The dose should be increased in 5 mg increments to 10 mg/day (5 mg twice daily), 15 mg/day (5 mg and 10 mg as separate doses), and 20 mg/day (10 mg twice daily). The minimum recommended interval between dose increases is one week. The dosage shown to be effective in controlled clinical trials is 20 mg/day. The contents of the U.S. FDA approved package inserts for the forgoing memantine products are incorporated herein by reference.

In some embodiments, memantine, or a pharmaceutically acceptable salt thereof, is 1-amino-3,5-dimethyladamantane hydrochloride.

In some embodiments, pharmaceutical compositions of the disclosure include memantine, i.e., memantine as a free base. In other aspects, pharmaceutical compositions of the disclosure include pharmaceutically acceptable salts of memantine. As used herein, amounts of memantine present in the pharmaceutical compositions of the disclosure refer to amounts of the memantine salt. For example, in those aspects wherein the pharmaceutical composition comprises a pharmaceutically acceptable salt of memantine, such as memantine hydrochloride, “10 mg of memantine” refers to 10 mg memantine hydrochloride, equivalent to 8.31 mg of memantine base in the pharmaceutical composition.

In some embodiments, lisinopril, or a pharmaceutically acceptable salt thereof, is present in the pharmaceutical composition in a subtherapeutic amount.

In some embodiments, lisinopril, or a pharmaceutically acceptable salt thereof, is present in the pharmaceutical composition in a therapeutic amount.

In some embodiments, the therapeutic amount of lisinopril, or a pharmaceutically acceptable salt thereof, is from about 5 to about 25 mg, administered once daily.

In some embodiments, the sub-therapeutic amount of lisinopril, or a pharmaceutically acceptable salt thereof, is less than about 5 mg, administered once daily.

In some embodiments, the pharmaceutical composition comprises about 1-40 mg of lisinopril, or a pharmaceutically acceptable salt thereof. In some embodiments, the pharmaceutical composition comprises about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39 or about 40 mg of lisinopril, or a pharmaceutically acceptable salt thereof.

In some embodiments, the memantine, or a pharmaceutically acceptable salt thereof, is present in the pharmaceutical composition in a subtherapeutic amount.

In some embodiments, the memantine, or a pharmaceutically acceptable salt thereof, is present in the pharmaceutical composition in a therapeutic amount.

In some embodiments, the therapeutic amount of memantine, or a pharmaceutically acceptable salt thereof, is from about 10 to about 30 mg, administered once daily.

In some embodiments, the subtherapeutic amount of memantine, or a pharmaceutically acceptable salt thereof, is less than about 10 mg, administered once daily.

In some embodiments, both the lisinopril and the memantine are present in the pharmaceutical composition in subtherapeutic amounts. In some embodiments, both the lisinopril and the memantine are present in the pharmaceutical composition in therapeutic amounts. In some embodiments, the lisinopril is present in the pharmaceutical composition in a subtherapeutic amount and the memantine in a therapeutic amount. In some embodiments, the lisinopril is present in the pharmaceutical composition in a therapeutic amount and the memantine in a subtherapeutic amount.

In some embodiments, the pharmaceutical composition comprises about 1-40 mg of memantine, or a pharmaceutically acceptable salt thereof. In some embodiments, the pharmaceutical composition comprises about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39 or about 40 mg of memantine, or a pharmaceutically acceptable salt thereof.

In some embodiments, the ratio of lisinopril, or a pharmaceutically acceptable salt thereof, to memantine, or a pharmaceutically acceptable salt thereof, present in the pharmaceutical composition is about 1:5 to about 5:1 w/w. In some embodiments, the ratio of lisinopril, or a pharmaceutically acceptable salt thereof, to memantine, or a pharmaceutically acceptable salt thereof, present in the pharmaceutical composition is about 1:5, about 1:4, about 1:3, about 1:2, about 1:1, about 2:5, about 2:4, about 2:3, about 2:1, about 3:5, about 3:4, about 3:2, about 3:1, about 4:5, about 4:3, about 4:1, about 5:4, about 5:3, about 5:2 or about 5:1 w/w.

In some embodiments, the ratio of lisinopril, or a pharmaceutically acceptable salt thereof, to memantine, or a pharmaceutically acceptable salt thereof, present in the pharmaceutical composition is about 1:3 to about 3:1 w/w.

In some embodiments, the ratio of lisinopril, or a pharmaceutically acceptable salt thereof, to memantine, or a pharmaceutically acceptable salt thereof, present in the pharmaceutical composition is about 1:1 w/w.

In some embodiments, the composition further comprises a pharmaceutically acceptable excipient. In some embodiments, the excipient is chosen from one or more disintegrants, binders, fillers and lubricants. Examples of disintegrants include, but are not limited to, agar, algin, calcium carbonate, carboxymethylcellulose, cellulose, clay, colloidal silicon dioxide, croscarmellose sodium, crospovidone, rubber, magnesium aluminum silicate, methylcellulose, polacrilin potassium, sodium alginate, low substituted hydroxypropyl cellulose, cross-linked polyvinyl pyrrolidone hydroxypropyl cellulose, sodium starch glycolate, and starch. Examples of binders include, but are not limited to, microcrystalline cellulose, hydroxymethylcellulose, hydroxypropylcellulose, and polyvinylpyrrolidone. Examples of fillers include, but are not limited to, one or more of calcium carbonate, calcium phosphate, dicalcium phosphate, tricalcium sulfate, carboxymethylcellulose calcium, cellulose, dextrate, dextrin, dextrose, fructose, lactitol, lactose, magnesium carbonate, magnesium oxide, methitol, maltodextrin, maltose, sorbitol, starch, sucrose, sugar, and xylitol. Examples of lubricants include, but are not limited to, one or more of agar, calcium stearate, ethyl oleate, ethyl laurate, glycerin, glyceryl palmitostearate, hydrogenated vegetable oil, magnesium oxide, magnesium stearate, mannitol, poloxamer, glycol, sodium benzoate, sodium lauryl sulfate, sodium stearyl, sorbitol, stearic acid, talc, and zinc stearate.

In some embodiments, the composition is formulated as a tablet, gel, oral liquid or syrup, capsule, suppository, injectable solution, inhalable form, nasal spray, or adhesive. In some embodiments, the composition is formulated as an immediate release oral dosage form. In other embodiments, the composition is formulated as an extended-release oral dosage form. In other embodiments, the composition is formulated as an immediate+extended-release oral dosage form. In other embodiments, the composition comprises a kit comprising immediate release oral dosage forms. In other embodiments, the composition comprises a kit comprising extended-release oral dosage forms. In other embodiments, the composition comprises a kit comprising immediate+extended-release oral dosage forms. In other embodiments, the composition comprises a kit comprising an immediate and an extended-release oral dosage form.

Also provided is a method of alleviating or preventing pain, wherein the pain is selected from migraine, tension-type headache, medication overuse headache, trigeminal autonomic cephalalgia (TAC), and trigeminal neuralgia, comprising administering to a subject in need thereof lisinopril, or a pharmaceutically acceptable salt thereof and memantine, or a pharmaceutically acceptable salt thereof. In some embodiments of the invention, the combined administration is of a pharmaceutical composition or kit as described herein. In some embodiments, the pain is selected from migraine, trigeminal autonomic cephalalgia (TAC) and trigeminal neuralgia.

Diagnosis or assessment of headache or pain is well-established in the art. References such as the International Classification of Headache Disorders, 3rd edition (ICHD-III; Cephalalgia 2018, Vol. 38 (1) 1-211) can be used by a skilled practitioner to assess the type of headache or pain experienced by a patient. Headaches within the scope of the instant invention include headaches of intracranial origin. Non-limiting examples of headaches of intracranial origin include migraine (e.g., chronic and episodic) and tension-type headaches. For example, “chronic migraine” refers to headache occurring on 15 or more days per month for more than three months, which has the features of migraine headache on at least 8 days per month, whereas “episodic migraine” refers to headache occurring less than 15 days per month, and “high frequency episodic migraine” refers to headache occurring between 8 and 14 days per month.

In some embodiments, the migraine is selected from episodic migraine, chronic migraine, high frequency episodic migraine, menstrual migraine, retinal migraine, migraine without aura, migraine with aura, familial hemiplegic migraine type 1 (FHM1), familial hemiplegic migraine type 2 (FHM2), familial hemiplegic migraine type 4 (FHM4), and sporadic hemiplegic migraine (SHM).

In some embodiments, the subject has an inadequate response to at least one medication used for the treatment of a migraine.

In some embodiments, the tension-type headache is selected from infrequent tension-type headaches, frequent tension-type headaches, chronic tension-type headaches and probable tension-type headaches.

In some embodiments, the subject has an inadequate response to at least one medication used for the treatment of a tension-type headache.

In some embodiments, the medication overuse headache is selected from headache associated with overuse of a specific medication. In some embodiments, the overuse is of ergotamine. In some embodiments, the overuse is of a triptan. In some embodiments, the overuse is of paracetamol. In some embodiments, the overuse is of a non-steroidal anti-inflammatory drug (NSAID). In some embodiments, the overuse is of a combination of specific medications.

In some embodiments, the subject has an inadequate response to at least one medication used for the treatment of a medication overuse headache.

In some embodiments, the TAC is selected from episodic cluster headache, chronic cluster headaches, paroxysmal hemicrania, hemicrania continua, short-lasting unilateral neuralgiform headache attacks with conjunctival injection and tearing (SUNCT), short-lasting unilateral neuralgiform headache attacks with cranial autonomic symptoms (SUNA), and long-lasting autonomic symptoms with hemicrania.

In some embodiments, the subject has an inadequate response to at least one medication used for the treatment of a TAC.

In some embodiments, the trigeminal neuralgia is classical trigeminal neuralgia. In some embodiments, the trigeminal neuralgia is secondary trigeminal neuralgia. In some embodiments, the trigeminal neuralgia is idiopathic trigeminal neuralgia.

In some embodiments, the classical trigeminal neuralgia is pure spontaneous trigeminal neuralgia or classical trigeminal neuralgia accompanied by persistent facial pain.

In some embodiments, the trigeminal neuralgia is painful trigeminal neuropathy.

In some embodiments, the painful trigeminal neuropathy is painful trigeminal neuropathy due to acute herpes zoster, postherpetic trigeminal neuropathy, idiopathic painful trigeminal neuropathy, painful posttraumatic trigeminal neuropathy, painful trigeminal neuropathy due to multiple sclerosis, painful trigeminal neuropathy due to space occupation lesions, or painful trigeminal neuropathy due to other diseases.

In some embodiments, the co-administration of the lisinopril, or a pharmaceutically acceptable salt thereof, and the memantine, or a pharmaceutically acceptable salt thereof, produces a comparable therapeutic effect with fewer side effects when compared to administration of either one alone.

In some embodiments, the co-administration of the lisinopril, or a pharmaceutically acceptable salt thereof, and the memantine, or a pharmaceutically acceptable salt thereof, results in an improved therapeutic index for either one of the lisinopril, or a pharmaceutically acceptable salt thereof, and the memantine, or a pharmaceutically acceptable salt thereof.

In some embodiments, the co-administration of the lisinopril, or a pharmaceutically acceptable salt thereof, and the memantine, or a pharmaceutically acceptable salt thereof, has a synergistic effect compared to either compound administered alone in the treatment of a pain selected from migraine, trigeminal autonomic cephalalgia. In some embodiments, the potency of the co-administration is about 5 times greater than lisinopril, or a pharmaceutically acceptable salt thereof, or memantine, or a pharmaceutically acceptable salt thereof, administered alone.

In some embodiments, the administration is for short-term treatment.

As used herein, the term “short-term treatment” refers to treating a subject for up to, and including, 7 days. Examples of conditions that might be treated by short-term treatment include menstrual migraine and acute migraine.

In some embodiments, the administration is for long-term treatment.

As used herein, the term “long-term treatment” refers to treating a subject for more than 7 days. Examples of conditions that might be treated by long-term treatment include chronic migraine and trigeminal neuralgias. On occasion, both short-term and long-term treatments can be preventative treatments.

In some embodiments, the administration is for acute treatment.

In some embodiments, the administration is for preventative treatment.

In some embodiments, the administration is for alleviating the pain.

In some embodiments, the administration is for preventing the pain.

In some embodiments, the administration is for reducing headache incidence.

In some embodiments, the reduction in headache incidence comprises a reduction from baseline in the number of mean monthly migraine days (MMDs), wherein a migraine day is defined as any day with migraine or probable migraine with or without aura according to the ICHD. In some embodiments, the reduction in headache incidence comprises a reduction from baseline in the number of mean monthly headache days (MHDs).

In some embodiments, the administration is for reducing the mean monthly acute medications for headache days, wherein an acute medication use day is defined as any day on which a study participant reports the intake of allowed medication(s) for the acute treatment of migraine.

In some embodiments, the administration is for ameliorating a headache or a symptom of a headache.

In some embodiments, the administration is for controlling a headache.

In some embodiments, the administration is for delaying the development or

progression of a headache.

In some embodiments, the administration results in a Responder rate of at least 50%. In some embodiments, the administration results in a Responder rate of 50%. In some embodiments, the administration results in a Responder rate of 50%, 55%, 60%, 65%, 70%, 75%, 80%, 85%, 90% or 95%.

Also provided is a kit as described herein for use in the treatment of pain, wherein the pain is selected from migraine, tension-type headache, medication overuse headache, trigeminal autonomic cephalalgia (TAC), and trigeminal neuralgia.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Embodiments of the present disclosure are provided in the following examples. These examples are presented only by way of illustration and to assist one of ordinary skill in using the disclosure. The examples are not intended in any way to otherwise limit the scope of the disclosure.

### Example 1. Reversing Effects of Lisinopril, Memantine and their Fixed Ratio (1:1) Combination on Face Allodynia in the Infraorbital Nerve Ligation Model in Rats

Infraorbital nerve ligation models, either by partial ligation or by the loose ligation of the whole nerve (chronic constriction injury) have been used as models of trigeminal neuralgia or trigeminal neuropathic pain. A partial infraorbital nerve ligation model in rats, based on eliciting a sustained hypersensitivity to tactile stimuli (“mechanical allodynia”) in the innervation field of the infraorbital nerve by an injury caused by partial ligation of the nerve, was used as a predictive model for evaluating the efficacy of lisinopril and memantine in the relief of trigeminal neuralgia, neuropathic pain, migraine and other headache disorders.

Male Sprague-Dawley rats weighing 200-220 g at surgery and 290-390 g at the time of testing were used. As primary read-outs of tactile pain sensitivity of the face, head withdrawal thresholds were measured by repeated stimulations using von Frey filaments (Touch Test Sensory Probes, Stoelting®). Before surgery, the rats were habituated to the testing for two weeks, first by handling (for 2 days) and then by repeated touching with von Frey filaments and finally determination of 50% head withdrawal thresholds (HWT) on three consecutive days. Rats with HWT<10 g at 2 out of 3 pre-surgery tests were not enrolled in the study. Sensitivity of the orofacial region was tested using calibrated von Frey filaments, with a series of filaments with bending force ranging between 0.4-14.8 g. The stimuli were applied near the center of the right whisker pad. Each filament was applied perpendicularly three times at approximately 1-sec intervals and pushed until it buckled. The response was considered positive if at least two out of the three touch stimulations elicited any of the following reactions: head withdrawal, face rubbing, grooming-like movements, headshaking, escape, or attack reaction. HWTs were determined using Chaplan's up-down method, starting with the filament of 2 g bending force.

For surgery, the animals were anesthetized with sodium pentobarbital (50 mg/kg i.p.). The right infraorbital nerve, just 1 to 2 mm rostral to its exit from maxilla through the infraorbital foramen, was exposed via a small skin incision, and approximately ⅓ to ½ portion of the nerve was dissected and tightly ligated with surgical suture (Polyamide monofil 6.0, Ethicon®). Then the skin incision was closed with surgical silk (Mersilk 5.0, Ethicon®). Rats were tested every second day during the first post-surgery week to follow the development of allodynia. Further measurements were performed on Days 9-11 post-surgery. Animals, in which 2 out of 3 tests did not show at least 50% reduction in HWT (animals with HWT higher than 9 g), were excluded from drug testing. From Day 12 up to the maximum of Day 40 post-surgery, eligible rats were repeatedly used for the drug tests, with at least 3-day washout periods kept between two testing days. On testing days, the animals were assigned to test groups receiving vehicle or different doses of a test substance, and HWT testing was performed pre-dose and at 30 min, 60 min and 120 min post-dose. Rats were administered with either lisinopril dihydrate or memantine hydrochloride or their combination at a 1:1 dose ratio, each at 3 or 4 dose levels per os by gavage at a dose volume of 5 ml/kg dissolved in physiological saline. Doses were calculated in terms of lisinopril anhydrate and memantine base, with correction factors of 1.09 and 1.20, respectively.

The evaluated post-dose data (HWTs in gram) were converted to percent reversals, taking the simultaneous post-dose average HWT value of the vehicle treated group as 0% and the pre-surgery average value of animals involved in the test substance-treated group as 100%. Percent individual maximum post-dose reversals were plotted in semi-logarithmic dose-response graphs and the effective doses causing 50% reversal of the allodynia (ED₅₀ values) for solo treatments, co-administration treatment and theoretical additive interaction values were calculated by sigmoidal curve fitting with Hill slope of 1 and bottom asymptote fixed at 0%.

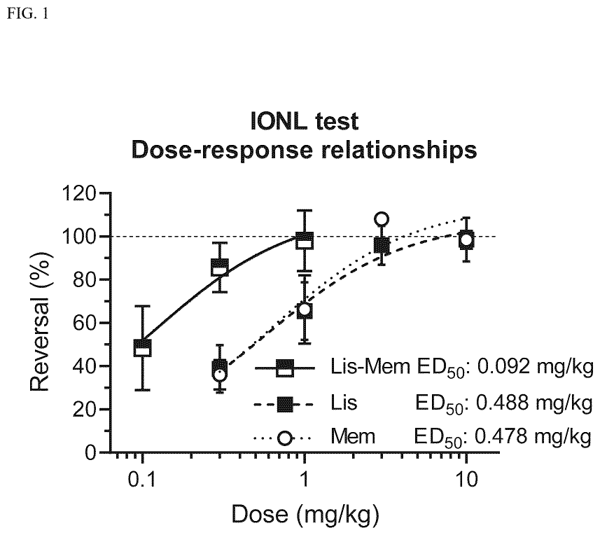

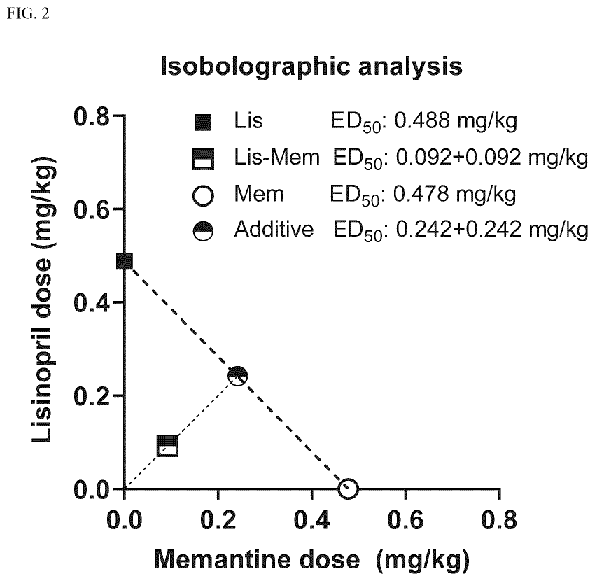

Each of the three test substances caused dose-dependent reversal of allodynia, which reached complete (about 100%) reversal at higher doses (FIG. 1). When in vivo potencies of lisinopril, memantine and their combination at 1:1 dose ratio were investigated, the mean (S.E.M.) ED₅₀ values for lisinopril and memantine were similar, 0.488 (0.141) mg/kg and 0.478 (0.137) mg/kg, respectively. However, the ED₅₀ value for the combination was unexpectedly low, 0.092 (0.017) mg/kg in terms of doses of the individual components, as compared to the theoretical additive ED₅₀ value of 0.242 (0.049), which was suggestive of a synergistic (supra-additive) interaction. As a result, approximately 5-fold lower doses of lisinopril and memantine were as effective in combination as the respective individual treatments. The calculated effective dose levels were plotted on an isobologram, where the isobole line represents the theoretical ED₅₀ values of additive interactions of dose combinations at different dose ratios, while the depicted ED₅₀ value of the 1:1 drug combination of the two components fell well below the isobole line (FIG. 2). This result indicates a supra-additive interaction between lisinopril and memantine. The difference between the observed and theoretical additive ED₅₀ values proved to be statistically significant (p<0.05) using a two-sided t-test.

### Example 2. Reversing Effects of Lisinopril, Memantine and their Fixed Ratio (1:1) Combination on Periorbital Allodynia in the Dural Inflammation Model in Rats

The dural inflammatory soup induced face allodynia (herein referred to as “Dural Inflammation”) model is a rat model of migraine in which sustained trigeminal sensitization is elicited by dural inflammation evoked by repetitive infusion of “inflammatory soup” on the dura mater (hard meninges) and allodynia is measured by periorbital pain threshold testing. This method was used to further investigate whether the unexpected synergy that was observed in the infraorbital nerve ligation model is also present in a different animal model which mimics migraine.

Male Sprague-Dawley rats weighing approximately 250 g at surgery were used. Rats were habituated and trained for 1-2 weeks and then fitted with a stainless-steel cannula (22GA, Plastics One) under isoflurane anesthesia. A craniotomy was performed using lambda as a reference (the junction of the superior sagittal sinus and transverse sinus) by advancing 1.5 mm right of lambda and 1 mm anterior towards bregma via a 1 mm hole that was made in the skull to expose the dura. Special attention was given not to disturb the dura. A custom flange guide cannula (22GA, Plastics One) was inserted into the hole (cut 0.5 mm below pedestal). The cannula was fixed to the bone with small screws and dental cement. A dummy that extended just past the end of the guide cannula was inserted to prevent scar tissue from forming, and thus clogging the cannula. Animals were allowed 1-2 weeks of recovery before testing and infusions began. Periorbital thresholds (POT) were monitored 7-10 days after surgery to ensure the thresholds generally returned to pre-surgery baselines. If animals did not return to historic baseline values (i.e., POT>6 g), they were excluded from the study.

While under isoflurane anesthesia, the infusion procedure proceeded over 5 consecutive days in which a Hamilton syringe attached to an infusion pump was used to deliver 15 μL of inflammatory soup over 8 minutes, at a rate of 1.759 μL/min. The Hamilton syringe was connected to tubing with a custom cannula injector at the tip; the syringe, tubing line, and injector were filled to the tip with inflammatory soup solution. The custom cannula injector was inserted into the flange cannula and the infusion started. The inflammatory soup contained 2 mM histamine, 2 mM bradykinin, 2 mM serotonin, and 0.2 mM prostaglandin E2 dissolved in saline.

After the 5-day inflammatory soup sensitization period, the rats had a one-week wash-out period and were then enrolled in the study. Only animals that developed sustained hypersensitivity and satisfied the periorbital sensitivity criteria (POT<2.5 g) were enrolled into the study and each week into test groups.

Sensory testing was performed on testing days using von Frey filaments (Stoelting®, Wood Dale, Illinois) with reproducible calibrated buckling forces varying from 0.4-10 g utilizing Chaplan's up-down method. The specific fibers utilized were 3.61 (0.4 g), 3.84 (0.6 g), 4.08 (1 g), 4.31 (2 g), 4.56 (4 g), 4.74 (6 g), 4.93 (8 g), and 5.07 (10 g); 4.31 (2 g) was the starting fiber. Allodynia was tested by gently restraining the rat in the investigator's hand by holding the rat in one hand with fingers just behind the left forepaw, keeping the animal pressed against the investigator's chest. Fibers were applied by perpendicularly touching the rostral portion of the periorbital region (below the lower eye margin close to the rostral corner) causing slight buckling of the filament for approximately 5 seconds. Based on the response pattern and the force of the final filament, the withdrawal threshold (g) was calculated.

On testing days, a blinded investigator tested periorbital sensitivity pre-dose, 45 min, 90 min, and 135 min after drug administration. At 0 min, animals were administered 0.1 mg/kg glyceryl trinitrate (GTN) intraperitoneally, followed immediately by per os administration of vehicle or test substances.

An interaction study of two drugs and their combination (i.e., 3 test substances) was performed over a 3-week period with 3 dose levels, i.e. with low, medium, and high doses examined at Week 1, Week 2, and Week 3, respectively. Thus, the study design with 3 test substances and 3 doses required forming 9 test substance-treated groups (Week 1: A1, B1, A+B1; Week 2: A2, B2, A+B2; Week 3: A3, B3, A+B3) and a vehicle-treated (V) control group. The animals administered a given test substance were re-used, after a 7-day washout at a higher dose. The animals were re-randomized each week, so that the group assignments were balanced in terms of periorbital sensitivity. Thus, the animals mostly did not receive the same test substance at higher dose during consecutive weeks. Each test group comprised 12 animals. Whereas the entire test substance treated groups were examined only during 2 days of a week, the vehicle-treated group was distributed over 3 weeks, i.e., 4 animals each week in parallel with the actual test substance groups.

In the study, lisinopril dihydrate and memantine hydrochloride (both purchased from MedChemExpress) were used. Doses were calculated in terms of the anhydrate and base, respectively. The dose levels were 0.2, 0.6 and 2 mg/kg for solo drug treatments and the corresponding 0.1+0.1, 0.3+0.3 and 1+1 mg/kg for the combination treatment at the low, medium, and high dose levels, respectively. The test substances were dissolved in distilled water at concentrations needed for dosing volumes of 5 mL/kg body weight and administered per os by gavage.

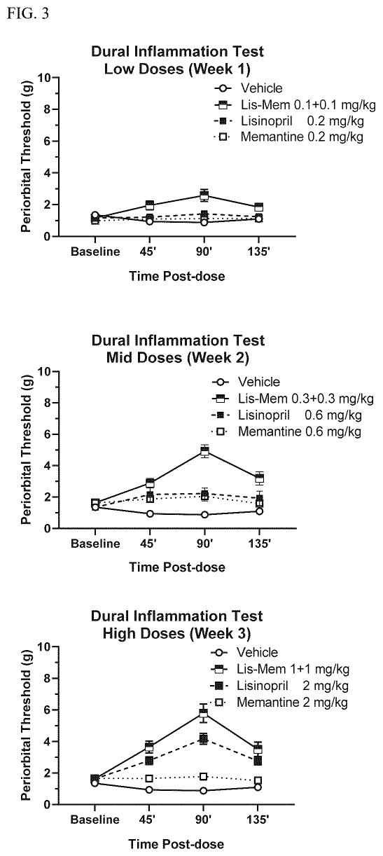

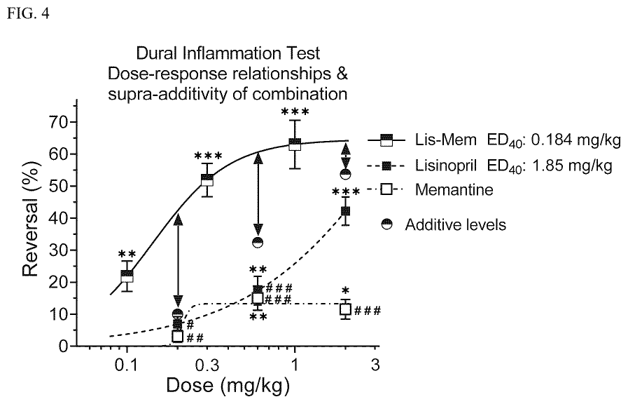

Data were expressed as the average±S.E.M. The average curve of time course of drug effects was plotted (FIG. 3) and the data at maximum allodynia reversing effects on these plots were selected for dose-response relationship determinations. The statistical significance of differences from corresponding vehicle data were evaluated by two-way repeated measures ANOVA followed by Dunnett's test. These test results are marked in the dose-response graph (FIG. 4) with symbols * p<0.05, ** p<0.01 and *** p<0.001. To test statistical significance of supra-additivity, Dunnett's test was also performed to compare the solo treatment data to the corresponding (low-, mid- and high-dose) data of the combination treatment as common control. These test results are marked in the dose-response graph (FIG. 4) with symbols # p<0.05, ## p<0.01 and ### p<0.001.

The evaluated post-dose data were converted to percent reversals taking the simultaneous (90 min) post-dose average POT value of the vehicle treated group (0.88 g) as 0% and the pre-surgery average value (8.67 g) of animals as 100%. Percent reversals were plotted in semi-logarithmic dose-response graphs and the effective doses causing 40% reversal of the allodynia (ED₄₀ values) were calculated by sigmoidal curve fitting (using GraphPad Prism 8.0 software) with the bottom asymptote fixed at 0% (FIG. 4).

Lisinopril and the combination with memantine (Lis-Mem 1:1) caused dose-dependent reversal of allodynia. However, memantine caused only a small (not exceeding 15%) reversing effect on the allodynia, though statistically significant at the medium and high dose levels (FIGS. 3 and 4). The combination treatment was more effective than the solo treatments at all three corresponding dose levels. At the medium dose level (0.6 mg/kg), the effects of lisinopril and memantine alone were of similar size, resulting in around 15% reversal. In the case of additive interaction, the corresponding dose of the combination (0.3+0.3 mg/kg) would have been expected to also yield about 15% reversal. However, the dose of the combination resulted in more than 50% reversal. The statistical evaluation indicated that the effects of solo treatments with memantine were statistically significantly lower than the respective treatments with the combination at all three dose levels (p<0.001). The effects of solo treatments with lisinopril were also statistically significantly lower than the respective treatments with the combination at least at the low and medium dose levels. As a result, the dose-response relationships indicated that the combination was equally effective to lisinopril at 10-fold lower doses, as indicated by ED₄₀ values of 0.184 mg/kg and 1.85 mg/kg, respectively.

These results indicate a surprisingly strong synergistic effect between lisinopril and memantine in the dural inflammation model in rats, which proved to be also statistically significant.

### Example 3. Pharmacokinetic Interaction Study of Lisinopril and Memantine in Rats

An improved therapeutic index cannot be expected if the apparent synergy was caused by a major pharmacokinetic interaction leading to a significant increase in the plasma concentrations of at least one of the drug components of the combination. Therefore, a pharmacokinetic interaction study was carried out in rats which compared the serum concentrations of lisinopril and memantine at different time points after administration at two dose levels within the dose-range that showed synergy in the migraine model experiments.

TABLE 1
Comparison of plasma concentrations between solo and combination treatments

| | Memantine | | Lis-Mem | | Memantine | | Lis-Mem | |
|---|---|---|---|---|---|---|---|---|
| Treatment | 0.3 mg/kg | | 0.3 + 0.3 mg/kg | | 1 mg/kg | | 1 + 1 mg/kg | |
| Time | Concentration of Memantine [ng/mL] | | | | | | | |
| [min] | Mean | S.D. | Mean | S.D. | Mean | S.D. | Mean | S.D. |
| 45 | 5.58 | 2.10 | 4.13 | 1.44 | 11.40 | 0.62 | 17.7** | 2.20 |
| 90 | 8.46 | 1.32 | 7.29 | 1.36 | 22.30 | 2.76 | 25.50 | 3.76 |
| 135 | 8.91 | 1.45 | 7.16 | 1.59 | 24.20 | 5.59 | 26.20 | 7.45 |
| | Lisinopril | | Lis-Mem | | Lisinopril | | Lis-Mem | |
| Treatment | 0.3 mg/kg | | 0.3 + 0.3 mg/kg | | 1 mg/kg | | 1 + 1 mg/kg | |
| Time | Concentration of Lisinopril [ng/ml] | | | | | | | |
| [min] | Mean | S.D. | Mean | S.D. | Mean | S.D. | Mean | S.D. |
| 45 | 5.75 | 0.88 | 5.56 | 0.28 | 9.86 | 0.08 | 9.58 | 1.87 |
| 90 | 9.44 | 0.40 | 9.91 | 1.84 | 43.00 | 10.50 | 35.30 | 13.40 |
| 135 | 14.20 | 2.68 | 14.60 | 3.97 | 57.00 | 11.30 | 55.60 | 19.80 |

**italic font: significant difference (p < 0.01)-combination vs. solo
Non-italic font: no significant difference (p > 0.05)

The statistical significance of differences between corresponding solo treatment and combination treatment mean values was evaluated by Student's unpaired t-test. The areas under the curve between TO (time of dosing) and last tested timepoint (135 min) were also calculated to assess the drug exposures in each animal (Table 2).

TABLE 2
Comparison of drug exposures (AUC_{0-135 min})

| | | Mean AUC | | | |
|---|---|---|---|---|---|
| | Dose | (ng · min/mL) | | % Diff | |
| | (mg/kg) | Solo | Combi | Combi vs. Solo | Significance |
| Lisinopril | 0.3 | 1002 | 1024 | 2.2 | N.S. |
| | 1 | 3663 | 3271 | −10.7 | N.S. |
| Memantine | 0.3 | 832 | 675 | −18.9 | N.S. |
| | 1 | 2063 | 2534 | 22.8 | N.S. |

Overall, the results showed no consistent or statistically or biologically significant differences in drug exposures as indicated by calculated AUC values, where percent differences between the corresponding solo and combination treatments were +2.2% and −10.7% for lisinopril, and −18.9% and +22.8% for memantine in the low- and high-dose groups respectively. Out of 12 comparisons for corresponding solo and combination dose levels only one incidental statistically significant difference was seen at 45 min post-dose for memantine at the higher (1 mg/kg) dose level.

### Example 4. Effects of Lisinopril, Memantine and their Fixed Ratio Combination on the Cardiovascular System Measured Using Telemetry in Awake Unrestrained Rats

The cardiovascular parameters of normotensive rats instrumented with telemetric devices were investigated to determine whether the observed in vivo pharmacodynamic synergy exists also for cardinal side effects of the drug components of the combination. As lisinopril is an antihypertensive drug, its vasodilator and antihypertensive effects or other related cardiovascular effects may be cardinal side effects. Therefore, a cardiovascular telemetry study was performed with lisinopril, memantine and their combination in rats.

Male Sprague-Dawley rats weighing 250-300 g at the start of the investigations were used. The animals were housed individually in artificially lightened chambers with a 12 h light and dark cycle (lights on at 07:00) and an ambient temperature of 20-22° C. Radiotelemetric units comprising transmitters with built in sensors (Data Sciences International, USA, Type: HD-S11) were surgically implanted into rats under sodium pentobarbital (50 mg/kg, i.p.) induced anesthesia. The rat abdomen was opened by a midline laparotomy. After visualizing the aorta, it was separated from the vena cava just caudal to the left dorsal muscular branch. A piece of 5-0 suture was placed between the vena cava and aorta so that the suture lied underneath the aorta. By this suture the aorta was temporarily occluded, and the catheter introduced into the vessel. After drying the aorta around the catheter entry site, a small amount of tissue adhesive was applied and 5-10 seconds later the suture was loosened. The bipolar ECG leads were tunneled subcutaneously into Lead II position and fixed with surgical sutures. Closure of the abdomen and skin was followed by 7 days recovery period. On drug testing days, blood pressure, electrocardiogram and body temperature were measured, and averages of systolic blood pressure, diastolic blood pressure, mean arterial blood pressure, heart rate and body temperature over every 1-min interval were calculated and recorded by the Dataquest A.R.T. data acquisition system on a personal computer.

Drug tests were performed on 8 rats used repeatedly according to a 4-way crossover design, carried out in three sessions. In each session, all the 8 animals were treated and tested on 4 occasions with 3- or 4-days washout periods between treatments (i.e. Mondays and Thursdays in two weeks), so that on each occasion 4 pairs of rats were treated with 4 types of treatment (vehicle control, test substance A, test substance B and combination A+B), and during the 4 occasions all the animals were given all the 4 types of treatment according to a Latin-square 4-way crossover design. In the three consecutive 2-week-long sessions, three dose levels of test substances were tested in an escalating order. Thus, in a 6-week-long study, effects of three types of treatments (A, B and A+B: lisinopril, memantine and the lisinopril-memantine combination) could be compared to the within-session control data group on the same animals, and this comparison was carried out at three dose levels: low, medium, and high, tested in sessions 1, 2 and 3, respectively.

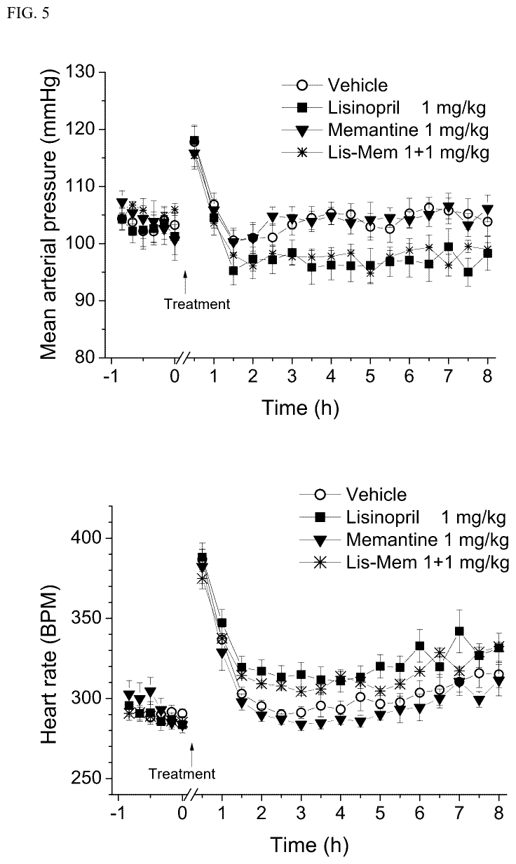

For evaluation and data reduction, pre-dose 10-min, post-dose 30-min segment averages were calculated and plotted (FIG. 5). The results showed that lisinopril, and the combination containing the same dose of lisinopril, decreased the blood pressure (mean, systolic and diastolic) and increased the heart rate of rats at all three dose levels, whereas memantine apparently did not have such effects. None of the test substance treatments caused any apparent effect on the body temperature as compared to vehicle control data. The stress of handling and oral gavage administration caused a transient increase in both blood pressure and heart rate as indicated by changes in the vehicle treated data. However, after these transient changes, blood pressure and heart rate returned to the pre-dose level in the vehicle control group. Since the lisinopril-induced changes were about maximal in the post-dose period of 3-6 hours, for further data reduction and quantitative evaluation average values for each animal in this period were calculated and subjected to statistical analysis and differences of mean±S.E.M. values from the within session vehicle mean values were plotted (Tables 3 and 4). For within session statistical analysis of differences from vehicle control, one-way ANOVA followed by Dunnett's test was used.

TABLE 3
Mean arterial blood pressure; difference from Vehicle treated (mmHg)

| Dose | Lisinopril | | | Memantine | | | Lis-Mem | | |
|---|---|---|---|---|---|---|---|---|---|
| (mg/kg) | Mean | S.E.M. | p | Mean | S.E.M. | p | Mean | S.E.M. | p |
| 0.3 | −7.05 | 1.17 | <0.001 | −1.39 | 1.70 | N.S. | −5.27 | 1.95 | <0.01 |
| 1 | −7.47 | 2.12 | <0.01 | 0.11 | 1.48 | N.S. | −6.60 | 1.93 | <0.05 |
| 3 | −7.72 | 0.92 | <0.001 | −0.13 | 1.17 | N.S. | −7.82 | 1.91 | <0.001 |

S.E.M.: Standard error of the mean
N.S.: Not significant (Dunnett's test)

TABLE 4
Heart rate; difference from Vehicle treated (beats per min)

| Dose | Lisinopril | | | Memantine | | | Lis-Mem | | |
|---|---|---|---|---|---|---|---|---|---|
| (mg/kg) | Mean | S.E.M. | p | Mean | S.E.M. | p | Mean | S.E.M. | p |
| 0.3 | 8.37 | 4.31 | N.S. | −2.87 | 5.41 | N.S. | 7.88 | 3.57 | N.S. |
| 1 | 20.62 | 5.49 | <0.01 | −8.51 | 3.11 | N.S. | 12.29 | 5.03 | N.S. |
| 3 | 29.39 | 1.59 | <0.001 | −3.03 | 3.38 | N.S. | 29.25 | 5.78 | <0.001 |

S.E.M.: Standard error of the mean
N.S.: Not significant (Dunnett's test)

The results indicate that lisinopril almost dose-independently suppressed the blood pressure by only about 7 mmHg, which is not considered a strong hypotensive effect. However, the heart rate was dose-dependently increased by lisinopril which indicates the degree of the vasodilating effects of drugs in normotensive rats. Overall, the compensating heart rate increase better reflects the drug's mild to moderate vasodilating effects than the blood pressure drops. Memantine alone had no effect on either hemodynamic parameter. The effects of the combination were similar but not greater at either dose level than those of lisinopril. Thus, it could be concluded that there was no positive interaction between lisinopril and memantine in terms of cardiovascular effects.

### Example 5. Effects of Lisinopril, Memantine and their Fixed Ratio Combination on the Central Nervous System Assessed in Rats by Locomotor Activity and Accelerating Rotarod Tests

Memantine is a neurological drug with neurological side effects at high doses, such as sedative or dissociative or psychotomimetic side effects in humans, and locomotion stimulating and motor coordination impairing effects in rodents. Therefore, as a further test to see if pharmacodynamic synergy exists also for cardinal side effects of the drug components of the combination two central nervous system side effect assessing pharmacodynamic interaction studies (locomotor activity and rotarod tests) were performed with lisinopril, memantine and their combination in rats.

Male Sprague Dawley rats weighing 200-250 g at start of testing were used. Animals were acclimatized to the laboratory conditions for at least 5 days before the first experiment and kept under artificial lighting between 7:00 and 19:00 in a controlled ambient temperature of 22±2° C. One hundred animals were assigned to ten treatment groups, which were tested in parallel with appropriate randomization, time shifts and cohorts needed to exploit the capacity of test apparatus. The test groups comprised nine test substance treated groups, of three treatment types (lisinopril, memantine and their 1:1 combination), each administered at three dose levels, and a vehicle control group for comparison. Corresponding dose levels of the combination were half of the doses of the solo drug treatments. Thus, the tested dose levels were 0.6, 2 and 6 mg/kg for the solo treatments and 0.3+0.3, 1+1 and 3+3 mg/kg for the combination. The test substances were dissolved in physiological saline for dosing volume of 5 mg/kg. The test substances were administered per os by gavage. First, the locomotor test was carried out. One week later, the same animals received the same treatment and were tested in the rotarod test.

The locomotor activity test detects stimulant or sedative activity. The activity meter consisted of 16 covered Plexiglas cages (40×25×25 cm) contained within a darkened enclosure and connected to silent electronic counters. Each cage was equipped with four photocell assemblies (two at each end of the cage) 3 cm above the floor to measure the number of movements by each animal (one per cage) in the horizontal plane. The number of (horizontal) crossings by each animal (one per cage) from one pair of photocells to the other was recorded by computer in 10-minute intervals. The animals were placed in the activity meter cages for 60 minutes, commencing 30 minutes after the administration of the test substance. The cumulated events in the period of 60-90 minutes post-dose were evaluated.

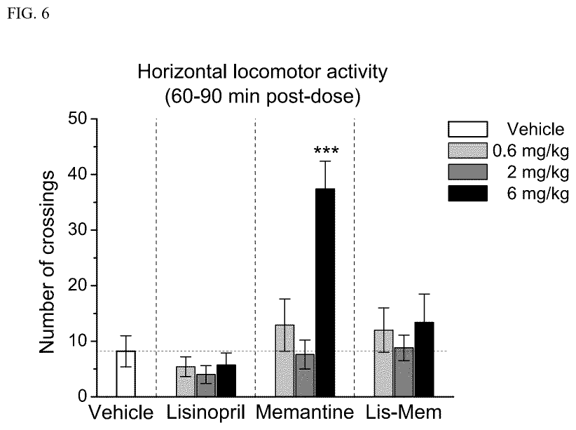

The results summarized in FIG. 6 show that neither lisinopril nor the combination exerted a significant effect on the locomotor activity of the rats at any of the three dose levels used. However, memantine at the highest dose level, but not at the two lower dose levels, caused a significant increase in the locomotor activity of the rats.

The rotarod test detects neurological deficits. Rats were placed on a rod (diameter: 7 cm) rotating at a constant speed of 4 revolutions per minute for a 2-minute period. When they fell off during this period they were replaced on the rod. This training procedure was repeated 2 times (i.e., 3 training sessions at a 5 min interval). Starting at least 2 hours after the last training sessions, rats were placed on the rotarod for a maximum period of 3 minutes. The rod rotated at a constant speed of 4 revolutions per minute at the beginning of the test and it progressively accelerated up to 30 revolutions per minute at the end of the test. The latency to fall off was recorded (cut-off: 3 minutes). The testing procedure was replicated twice with 5 min interval at two post-dose occasions: first 45 min post-dose, next 90 minutes post-dose.

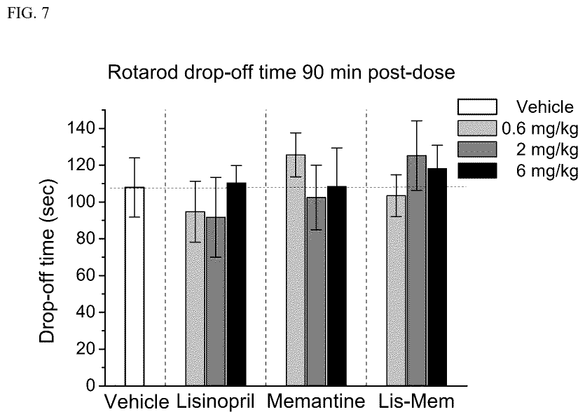

The results summarized in FIG. 7 show that neither lisinopril, nor memantine, nor their combination had a significant effect on rat rotarod performance at any of the three dose levels used, suggesting no significant sedative or motor coordination impairing effects, or any apparent synergy for these type of effects at doses up to 3 times the top dose used in the Dural Inflammation Migraine Modeling Test.

### Example 6: Lack of Synergy for Candesartan-Memantine Fixed Ratio (1:1) Combination in the Dural Inflammation Model in Rats

Candesartan is a representative member of angiotensin II receptor blockers, which inhibit the effects angiotensin and are known to have some therapeutic value in prophylactic treatment of migraine. The presence or absence of synergy between candesartan and memantine was investigated in the dural inflammation test as described in Example 2, using the same study design, except that the applied drugs were candesartan cilexetil (purchased from MedChemExpress) and memantine hydrochloride (purchased from Merck/Supelco). Candesartan cilexetil, a prodrug, is hydrolyzed to the candesartan active form in the gastrointestinal tract during absorption.

In the candesartan-memantine combination study, the dose levels were 0.2, 0.6 and 2 mg/kg for solo drug treatments and the corresponding 0.1+0.1, 0.3+0.3 and 1+1 mg/kg for the combination treatment at the low, medium, and high dose levels, respectively. The test substances were suspended in a vehicle of 0.5% Methylcellulose+0.2% tween 80 dissolved in distilled water at concentrations needed for dosing volumes of 5 mL/kg body weight and administered per os by gavage.

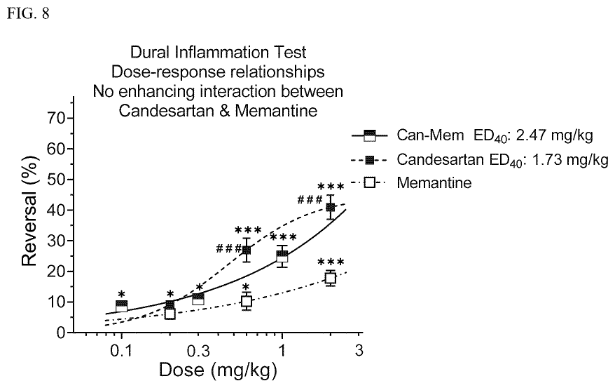

Following the evaluation procedure described in Example 2, percent reversals were plotted in semi-logarithmic dose-response graphs and the effective doses causing 40% reversal of the allodynia (ED₄₀ values) were calculated by sigmoidal curve fitting (using GraphPad Prism 8.0 software) with the bottom asymptote fixed at 0% (FIG. 8). The statistical significance of differences from corresponding vehicle data were evaluated by two-way repeated measures ANOVA followed by Dunnett's test. These test results are marked in the dose-response graph (FIG. 8) with symbols * p<0.05, ** p<0.01 and *** p<0.001. To test statistical significance of deviations from additivity, Dunnett's test was also performed to compare the solo treatment data to the corresponding (low-, mid- and high-dose) data of the combination treatment as common control. These test results are marked in the dose-response graph (FIG. 8) with symbols # p<0.05, ## p<0.01 and ### p<0.001.

The results showed that the effective doses of combination in terms of candesartan doses were similar or slightly less effective than with candesartan alone, indicating an infra-additive interaction between candesartan and memantine. These results suggest that the surprising synergy observed between lisinopril and memantine is not a feature that can be extended to the interaction of memantine with all types of angiotensin inhibitors or antimigraine drugs.

### Example 7: The Effects of Lisinopril, Memantine and their Fixed Ratio (1:1) Combination on Face Allodynia in the Repeat Dose Infraorbital Nerve Ligation Model in Rats

Example 1 demonstrated a strong synergistic effect between lisinopril and memantine in the rat partial infraorbital nerve ligation model, which is considered a chronic model of migraine or trigeminal neuralgia or trigeminal neuropathic pain. The protocol of Example 1 was adapted to investigate the effects of lisinopril and memantine following repeated dosing.

40 male Sprague-Dawley rats were anesthetized with sodium pentobarbital (50 mg/kg i.p.). The right infraorbital nerve rostral to its exit from maxilla was exposed and approximately ⅓ to ½ portion of the nerve was dissected and tightly ligated. As primary read-outs of tactile pain sensitivity of the face, head withdrawal thresholds (HWT) were measured by repeated stimulations using von Frey filaments. A series of filaments with bending force ranging between 0.4-14.8 g was used. The stimuli were applied near the center of the right whisker pad. Each filament was applied perpendicularly three times at approximately 1-sec intervals and pushed until it buckled. The response was considered positive if at least two out of the three touch stimulations elicited any of the following reactions: head withdrawal, face rubbing, grooming-like movements, headshaking, escape or attack reaction. HWTs were determined using Chaplan's up-down method (Chaplan et al., 1994), starting with the filament of 2 g bending force.

HWT measurements were performed before surgery, and animals with HWT<10 g at 2 out of 3 pre-surgery tests were not subjected to surgery. On Day 4, Day 6, Day 8 and Day 11 post-surgery, HWTs were tested to observe development of allodynia. Only the animals showing stable allodynia with HWT<5 g on Day 12 pre-dose were enrolled in the study and randomized into two treatment groups with equalized starting pre-dose average HWT of 3.6 g.

Starting on Day 12 post-surgery (Day 1 of treatment), groups of 9 and 10 rats were administered per os b.i.d. for 10 days with either vehicle (saline 5 ml/kg) or lisinopril and memantine (1+1 mg/kg), respectively. On each treatment day, a morning dose was administered around 7:00 a.m. and an afternoon dose around 2:00 p.m. On the treatment days of Day 1, Day 2, Day 3, Day 5, Day 7 and Day 10, HWT measurements were performed before and 60 minutes after the morning dose.

In the vehicle group, stable allodynia was detected, average HWT remained in the range of 1.8-3.6 g, without any consistent change between pre- and post-dose. However, in the combination group, already the first dose of lisinopril and memantine caused significant reversal of allodynia to an average of HWT 9.4 g. On the second treatment day the pre-dose average HWT (10 g) indicated a persisting anti-allodynic effect despite 17 hours having passed since the previous dose. Nevertheless, after the dosing, the average HWT increased further to 13 g. During the subsequent testing days, the pre- and post-dose HWT values gradually increased further, but a small step-up from pre-dose to post-dose was always present. On Day 10 of treatment, complete reversal of allodynia was reached, as the average HWT was equivalent with the pre-surgery control value (15.7 g) of the same group. The difference between lisinopril and memantine and vehicle became statistically significant after the first dosing (p<0.01) and remained significant (p<0.001) throughout the treatment period at both pre- and post-dose measurements of HWT (repeated measures ANOVA followed by Bonferroni's post-hoc test).

These results demonstrate that no tolerance develops to the anti-allodynic effect of a lisinopril and memantine combination upon repeated dosing. Instead, repeated dosing twice daily elicits a sustained allodynia reversing effect that is nearly 80% reversal by Day 5 of treatment pre-dose, and 100% reversal after dosing on Day 10. These data suggest that the combination may be used for preventive treatment of episodic or chronic migraine and other headache disorders. The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of treating or preventing pain comprising administering to a subject in need thereof, a pharmaceutical composition comprising a combination of lisinopril, or a pharmaceutically acceptable salt thereof, and memantine, or a pharmaceutically acceptable salt thereof and wherein the pain is selected from migraine, tension-type headache, medication overuse headache, trigeminal autonomic cephalalgia or trigeminal neuralgia.

2. The method of claim 1, wherein the migraine is selected from episodic migraine, chronic migraine, high frequency episodic migraine, menstrual migraine, retinal migraine, migraine without aura, migraine with aura, familial hemiplegic migraine type 1 (FHM1), familial hemiplegic migraine type 2 (FHM2), familial hemiplegic migraine type 4 (FHM4) or sporadic hemiplegic migraine (SHM).

3. The method of claim 1, wherein the tension-type headache is selected from infrequent tension-type headaches, frequent tension-type headaches, chronic tension-type headaches or probable tension-type headaches.

4. The method of claim 1, wherein the medication overuse headache is selected from ergotamine overuse, triptan overuse, paracetamol overuse, NSAID overuse or a combination thereof.

5. The method of claim 1, wherein the trigeminal autonomic cephalalgia is selected from episodic cluster headache, chronic cluster headaches, paroxysmal hemicrania, hemicrania continua, short-lasting unilateral neuralgiform headache attacks with conjunctival injection and tearing (SUNCT), short-lasting unilateral neuralgiform headache attacks with cranial autonomic symptoms (SUNA) or long-lasting autonomic symptoms with hemicrania.

6. The method of claim 1, wherein the trigeminal neuralgia is selected from classical trigeminal neuralgia, secondary trigeminal neuralgia, painful trigeminal neuropathy or idiopathic trigeminal neuralgia.

7. The method of claim 1, wherein the subject has an inadequate response to at least one medication used for the treatment of pain.

8. The method of claim 1, wherein the administration is for acute treatment.

9. The method of claim 1, wherein the administration is for preventative treatment.

10. The method of claim 1, wherein the pharmaceutical composition comprises a sub-therapeutic amount of lisinopril, or a pharmaceutically acceptable salt thereof and/or of memantine, or a pharmaceutically acceptable salt thereof.

11. The method of claim 1, wherein the pharmaceutical composition comprises a therapeutic amount of lisinopril, or a pharmaceutically acceptable salt thereof and/or of memantine, or a pharmaceutically acceptable salt thereof.

12. The method of claim 1, wherein the pharmaceutical composition comprises about 1 to about 40 mg of lisinopril, or a pharmaceutically acceptable salt thereof.

13. The method of claim 1, wherein the pharmaceutical composition comprises about 1 to about 40 mg of memantine, or a pharmaceutically acceptable salt thereof.

14. The method of claim 1, wherein the pharmaceutical composition comprises lisinopril, or a pharmaceutically acceptable salt thereof and memantine, or a pharmaceutically acceptable salt thereof, in a ratio of about 1:5 to about 5:1 w/w.

15. The method of claim 14, wherein the ratio is of about 1:3 to about 3:1 w/w.

16. The method of claim 14, wherein the ratio of about 1:1 w/w.

17. The method of claim 1, wherein the pharmaceutical composition is a tablet, gel, oral liquid, syrup, capsule, suppository, injectable solution, inhalable form, nasal spray or an adhesive.

18. The method of claim 1, wherein the pharmaceutical composition comprises a kit comprising a first container and a second container, the first container comprising lisinopril, or a pharmaceutically acceptable salt thereof, and the second container comprising memantine, or a pharmaceutically acceptable salt thereof.

19. A method of treating migraine, tension-type headache, medication overuse headache, trigeminal autonomic cephalalgia or trigeminal neuralgia, comprising administering to a subject in need of treatment thereof, a pharmaceutical composition comprising a combination of lisinopril, or a pharmaceutically acceptable salt thereof, and memantine, or a pharmaceutically acceptable salt thereof.

20. A method of preventing migraine, tension-type headache, medication overuse headache, trigeminal autonomic cephalalgia or trigeminal neuralgia comprising administering to a subject in need thereof, a pharmaceutical composition comprising a combination of lisinopril, or a pharmaceutically acceptable salt thereof, and memantine, or a pharmaceutically acceptable salt thereof.
